(19) United States
(12) Patent Application Publication
(10) Pub. No.: US 2016/0257650 A1
(43) Pub. Date: Sep. 8, 2016

(54) MONOARYLATION OF AROMATIC AMINES

(71) Applicant: UMICORE AG & CO. KG, Hanau-Wolfgang (DE)

(72) Inventors: Lukas GOOSSEN, Kaiserslautern (DE); Matthias GRUENBERG, Schifferstadt (DE)

(73) Assignee: Umicore AG & Co. KG, Hanau-Wolfgang (DE)

(21) Appl. No.: 15/030,639

(22) PCT Filed: Oct. 17, 2014
(86) PCT No.: PCT/EP2014/072332

§ 371 Date: Apr. 20, 2016

(30) Foreign Application Priority Data
Oct. 21, 2013 (DE) 10 2013 017 369.8
Jan. 27, 2014 (EP) 14152698.8
Feb. 13, 2014 (EP) 14155079.8

(51) Int. Cl. C07D 209/86; C07D 409/12; B01J 31/22; C07C 221/00; C07F 17/02; B01J 31/24; C07D 405/12; C07C 209/10

(52) U.S. Cl. CPC C07D 209/86; C07D 405/12; C07D 409/12; C07C 209/10; C07C 221/00; C07F 17/02; B01J 31/2409; B01J 31/2295; C07C 2103/18; C07C 2103/24; B01J 2231/44; B01J 2531/842; B01J 2531/824

(57) ABSTRACT

The present invention relates to a selective process for performing the Hartwig-Buchwald coupling of biphenyl derivatives.

The Hartwig-Buchwald reaction has over time entered industrial manufacturing processes, particularly for intermediates in the fabrication of organic light-emitting diodes.

EP-A1-2407502 describes the preparation of dendrimers for this purpose, obtaining secondary amine intermediates through the Hartwig-Buchwald reaction.

EP-A2-2421064 as well makes use of the Hartwig-Buchwald reaction as a synthesis pathway for secondary amines which are utilized as intermediates.

The mediocre yield is a drawback, but it is the lack of selectivity that represents the greater problem, leading not only to secondary but also to tertiary amine by-products. These by-products are difficult to remove by sublimation. A particular drawback here, moreover, is that the purity requirements imposed on intermediates for preparing dendrimeric intermediates in the fabrication of organic light-emitting diodes are particularly exacting, given that unwanted impurities, even structurally related impurities, and even in small amounts, can lead to an unwanted shift in the emission wavelengths or reduction in the quantum yield, and hence also to unwanted evolution of heat.

It is the object of the present patent application to provide a process which permits the preparation of dibisphenylamines under the conditions of Hartwig-Buchwald coupling in relatively high yields and selectivities, allowing a reduction in the cost and complexity associated with purifying the compounds, which is done usually by sublimation. In light of the above explanation, then, it is necessary to achieve the increased yield primarily through an increase in the selectivity.

The object is achieved by a process for selective arylation of a primary aromatic amine of the formula A-NH₂ with an aromatic compound of the formula X—B to give a secondary aromatic amine A-NH—B, the radicals A and B independently of one another being identical or different, substituted or unsubstituted aromatic radicals and the radical X being a leaving group, more particularly halogen, i.e. fluorine, chlorine, bromine, iodine or astatine, or a trifluoromethylsulphonic acid radical, the aromatic carbon atoms in the secondary aromatic amine being bonded directly to the nitrogen atom, and at least one of the radicals, A or B, comprising a biphenyl unit, and the reaction being carried out in the presence of a base and a palladium complex, the palladium atom being complexed by a bis(dialkylphosphinoferrocene) ligand.

### BRIEF DESCRIPTION OF THE INVENTION

The invention may be described briefly in the following points:
1. Process for selective arylation of a primary aromatic amine of the formula A-NH₂ with an aromatic compound of the formula X—B to give a secondary aromatic amine A-NH—B, the radicals A and B independently of one another being identical or different, substituted or unsubstituted aromatic radicals and the radical X being a halogen or a trifluoromethylsulphonic acid radical, the aromatic carbon atoms in the secondary aromatic amine being bonded directly to the nitrogen atom, and at least one of the radicals, A or B, comprising a biphenyl unit, and the reaction being carried out in the presence of a base and a palladium complex, the palladium atom being complexed by at least one bis(dialkylphosphinoferrocene) ligand.
2. Process according to point 1, the alkyl substituents of the bis(dialkylphosphinoferrocene) ligands having two to five carbon atoms.
3. Process according to point 1 or 2, the alkyl substituents being selected from the group consisting of isopropyl, isobutyl, tert-butyl and combinations thereof.
4. Process according to any of points 1 to 3, both aromatic substituents, A and B, comprising a biphenyl unit, these units being identical to or different from one another.
5. Process according to any of points 1 to 4, the biphenyl unit being bonded directly to the secondary nitrogen atom of the amine.
6. Process according to any of the preceding points, the alkyl substituents being selected from the group consisting of isopropyl, isobutyl and combinations thereof.
7. Process according to any of the preceding points, the biphenyl unit being bonded directly to the leaving group, more particularly to a halogen, i.e. chlorine, bromine or Iodine, or to a trifluoromethyl-sulphonic acid group.
8. Process according to any of the preceding points, the biphenyl unit being a bridged biphenyl unit of the formula 2 or 3

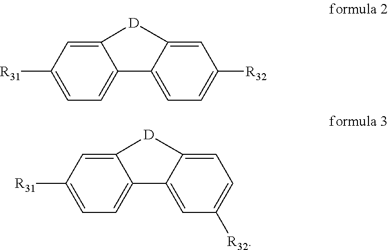

where D may be oxygen, sulphur, nitrogen or carbon and may be substituted in the case of nitrogen singly or in the case of carbon doubly by methyl, ethyl, biphenyl, naphthyl or phenyl.
9. Process according to one or more of the preceding points, the biphenyl unit A, B or both being identical or different and being selected from a unit of the formulae

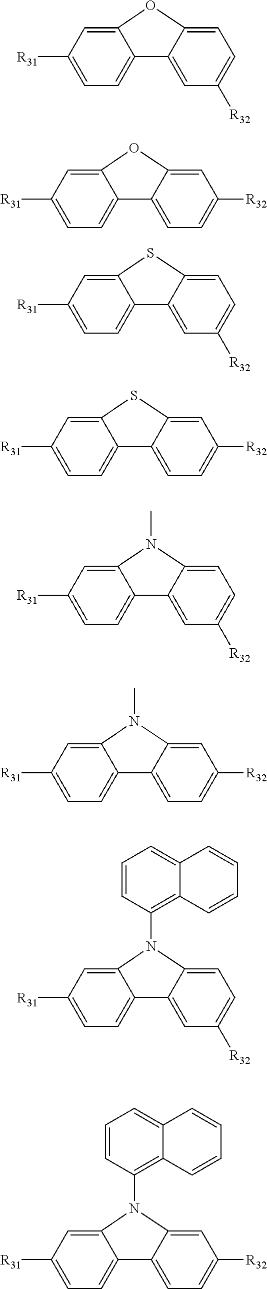

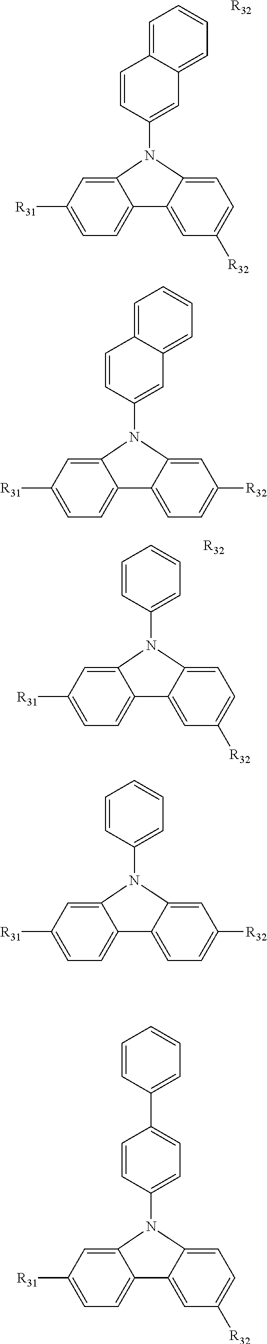

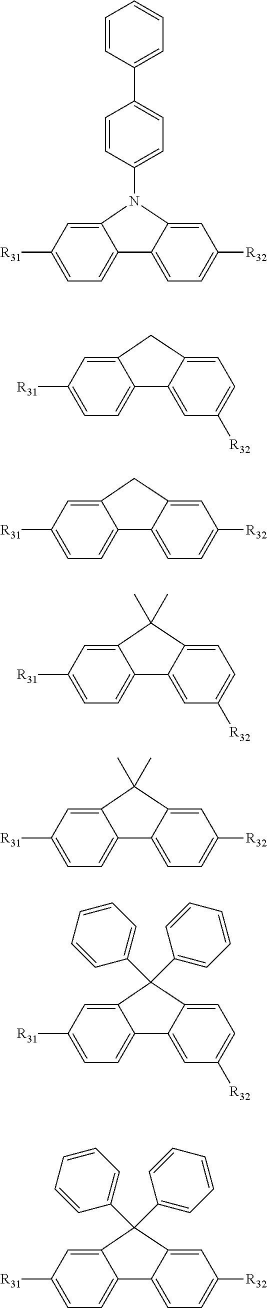

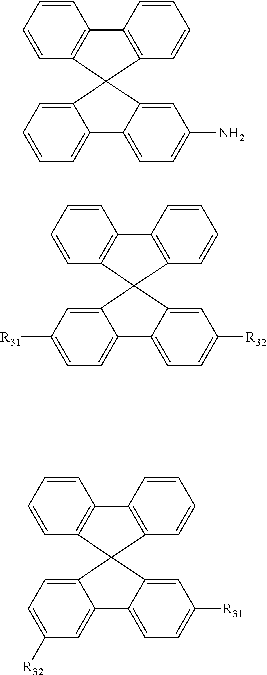

where R31 may be hydrogen, phenyl, biphenyl or pyridyl,
R32 may be a leaving group, halogen, a primary amine group NH₂ or a trifluoromethylsulphonic acid radical, depending on whether the radical is used as A or B; or else R32 is a spacer which is arranged between A or B and X or NH₂, respectively.
10. Process according to one or more of the preceding points, the spacer being selected from the group consisting of 1,4-phenyl, 1,4-(6-methyl)phenyl, 1,4-(5-methyl)phenyl, 4,4′-biphenyl, 2,6-naphthyl, 1,4-naphthyl,

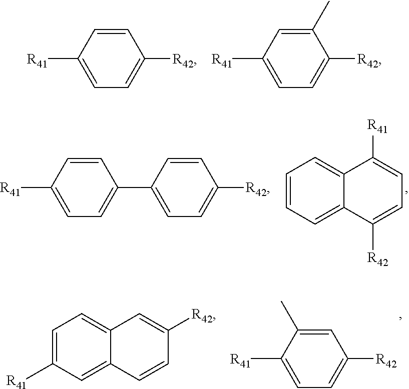

R41 here is a structure of the formulae 2 to 22, R42 may be halogen such as fluorine, chlorine, bromine, iodine or astatine, a primary amine group NH₂ or a trifluoromethylsulphonic acid radical.
11. Process according to one or more of the preceding points, the base being lithium hydroxide, potassium hydroxide, sodium hydroxide, tertiary organic amines, alkoxides, tributylamine, triethylamine, alkali metal alcoholates, lithium ethanolate, sodium ethanolate, potassium ethanolate, lithium tert-butanolate, sodium tert-butanolate, potassium tert-butanolate, lithium bis(trimethylsilyl)amide, sodium bis(trimethylsilyl)amide, potassium bis(trimethylsilyl)amide or combinations thereof.
12. Process according to one or more of the preceding points, the process being carried out in a solvent selected from the group consisting of alcohols, ethanol, propanol, isopropanol, butanol, isobutanol, ethylene glycol, ethers, diethyl ether, tert-butyl ethyl ether, tert-butyl methyl ether, tetrahydrofuran, dioxane, ethylene glycol dimethyl ether, bis(2-methoxyethyl) ether, aromatic solvents, benzene, toluene, xylene, o-xylene, p-xylene, m-xylene and combinations thereof.
13. Process according to one or more of the preceding points, the process being carried out at a temperature of 5° C. to 150° C., 20° C. to 140° C., 30° C. to 130° C., 60° C. to 120° C. or 70° C. to 111° C.
14. Process according to one or more of the preceding points, the reaction time of the process being from one hour to 36 hours, 4 hours to 24 hours, 6 hours to 16 hours or 8 hours to 12 hours.
15. Process according to one or more of the preceding points, the biphenyl unit being 2-fluorene, 3-fluorene, 2-(9,9-diphenylfluorene), 2-(9,9-dimethylfluorene), 3-(9,9-diphenylfluorene), 3-(9,9-dimethylfluorene), 3-(4-phenyl)-9-phenyl-9H-carbazole, 3-(4-phenyl)-9-methyl-9H-carbazole, 3-(4-phenyl)-9-biphenyl-9H-carbazole, 2-(4-phenyl)-9-phenyl-9H-carbazole, 2-(4-phenyl)-9-methyl-9H-carbazole, 2-(4-phenyl)-9-biphenyl-9H-carbazole, 3-(4-biphenyl)-9-phenyl-9H-carbazole, 3-(4-biphenyl)-9-methyl-9H-carbazole, 3-(4-biphenyl)-9-biphenyl-9H-carbazole, 2-(4-biphenyl)-9-phenyl-9H-carbazole, 2-(4-biphenyl)-9-methyl-9H-carbazole, 2-(4-biphenyl)-9-biphenyl-9H-carbazole, 3-(9-phenyl-9H-carbazole), 3-(9-methyl-9H-carbazole), 3-(9-biphenyl-9H-carbazole), 2-(9-phenyl-9H-carbazole), 2-(9-methyl-9H-carbazole), 2-(9-biphenyl-9H-carbazole) or triphenylene.
16. Process according to one or more of the preceding points, the palladium complex being used in amounts of 0.01 mol % to 1.5 mol %, based on the total molar amount of both reactants, of the primary aromatic amine A-NH₂ and of the haloaromatic X—B.
17. Process according to one or more of the preceding points, the palladium complex being used as a solid, solution or powder mixture with a solid bis(dialkylphosphinoferrocene).
18. Process according to any of the preceding points, comprising the steps of:
   providing the primary aromatic amine A-NH₂, the haloaromatic X—B, a suitable solvent and optionally a bis(dialkylphosphinoferrocene) in a reaction vessel;
   adding a palladium complex in which the palladium atom is complexed by at least one bis(dialkylphosphinoferrocene) ligand, in the form of a solid or a solution;
   heating the resulting reaction mixture in the reaction vessel;
   Isolating the reaction product, a secondary aromatic amine A-NH—B; and
   optionally purifying the secondary aromatic amine A-NH—B.
19. Process according to one or more of the preceding points, the compounds

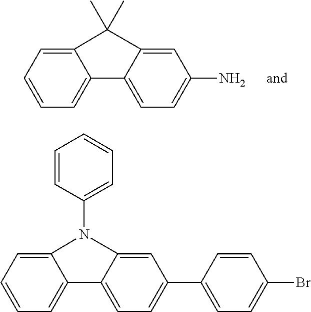

being present.
20. Process according to one or more of the preceding points, the palladium atom in the palladium complex being complexed additionally by 2,4,6,8-tetramethylcyclotetrasiloxane, bis(dibenzylideneacetone) or maleimide.
21. Process according to one or more of the preceding points, the palladium atom in the palladium complex being complexed by 1,1′-bis(diisopropylphosphino)ferrocene.
22. The chemical compound 1,1′-bis(dlisopropylphosphino)ferrocene-palladium(0)-maleimide [Pd(dippf)(maleimide)]; 1,1′-bis(diiso-propylphosphino)ferrocenepalladium(0)-1,3-divinyl-1,1,3,3-tetramethyldisiloxane [Pd(dippf)(VTS)]; 1,1′-bis(diisopropyl-phosphino)ferrocenepalladium(0)-bis(dibenzylideneacetone) [Pd(dippf)(dba)]; 1,1′-bis(diisopropylphosphino)ferrocene-palladium(0)-naphthoquinone [Pd(dippf)(naphthoquinone)]; 1,1′-bis(dlisopropylphosphino)ferrocenepalladium(0)-maleic anhydride [Pd(dippf)(maleic anhydride)]; 1,1′-bis(diisopropyl-phosphino)ferrocenepalladium(0)-diethyl maleate [Pd(dippf)(diethyl maleate)]; or 1,1′-bis(diisopropylphosphino)ferrocenepalladium(0)-norbornene [Pd(dippf)(norbornene)].
23. Powder mixture comprising a bis(dialkylphosphinoferrocene) and a palladium complex in which the palladium atom is complexed by at least one bis(dialkylphosphinoferrocene) ligand.
24. Use of a compound according to point 22 or of a powder mixture according to point 23 as catalyst for Hartwig-Buchwald coupling.

### DETAILED DESCRIPTION OF THE INVENTION

The palladium atom in the palladium complex is complexed by at least one bis(dialkylphosphinoferrocene) ligand which has the general formula 1:

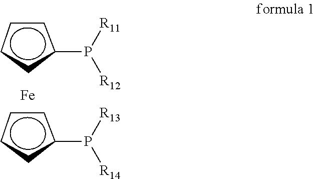

R11 to R14 may be identical or different and are, in particular, alkyl radicals having one to five carbon atoms. R11 to R14 may therefore, independently of one another, be selected from methyl, ethyl, n-propyl, isopropyl, n-butyl, isobutyl, sec-butyl, tert-butyl, n-pentyl (amyl), 2-pentyl (sec-pentyl), 3-pentyl, 2-methylbutyl, 3-methylbutyl (isopentyl or isoamyl), 3-methylbut-2-yl, 2-methylbut-2-yl, 2,2-dimethylpropyl (neopentyl).

Advantageously, R11 to R14 are identical and selected from the group consisting of methyl, ethyl, n-propyl, isopropyl, n-butyl, isobutyl, sec-butyl, tert-butyl, n-pentyl (amyl), 2-pentyl (sec-pentyl), 3-pentyl, 2-methylbutyl, 3-methylbutyl (isopentyl or isoamyl), 3-methylbut-2-yl, 2-methylbut-2-yl, 2,2-dimethylpropyl (neopentyl).

Particularly suitable are isopropyl, isobutyl, tert-butyl, more particularly isopropyl and tert-butyl. Good results are obtained when R11 to R14 are identical and are isopropyl or tert-butyl. Good results are obtained in particular when R11 to R14 are identical and are isopropyl, propyl or isobutyl. Good results are obtained in particular when R11 to R14 are identical and are isopropyl.

It has surprisingly been found that bis(dlalkylphosphinoferrocene) ligands of the formula 1, in the preparation of secondary amines by Hartwig-Buchwald coupling, allow the selective preparation for secondary amines with high yields if at least one of the radicals, A or B, in the compounds for coupling has at least one biphenyl unit as a structural element. In this case, the amount of palladium used may be lowered from the 3 mol % normally employed to around 1.5 mol % or less; the double arylation that normally occurs as a secondary reaction, forming the tertiary amine, is suppressed, and the reaction proceeds to the desired product with conversions of generally 90% or more.

With preference, both radicals A and B have a biphenyl unit. The biphenyl unit may be unsubstituted or substituted, including by one or more phenyl radicals, thus forming, for example, terphenyl, quaterphenyl or triphenylene units, which may be substituted or unsubstituted. The biphenyl units may also be bridged, as in the case of fluorene and its derivatives, for example. As biphenyl it is possible in particular to use the compounds of the formulae 2 or 3 as radicals A or B:

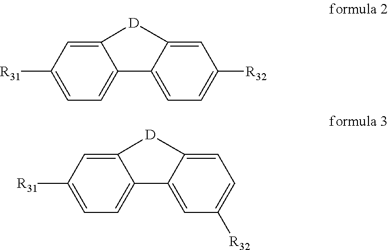

D may be oxygen, sulphur, nitrogen or carbon and may be substituted singly (in the case of nitrogen) or doubly (in the case of carbon) by methyl, ethyl, biphenyl, 1-naphthyl, 2-naphthyl or phenyl. The following radicals in particular may therefore be present:

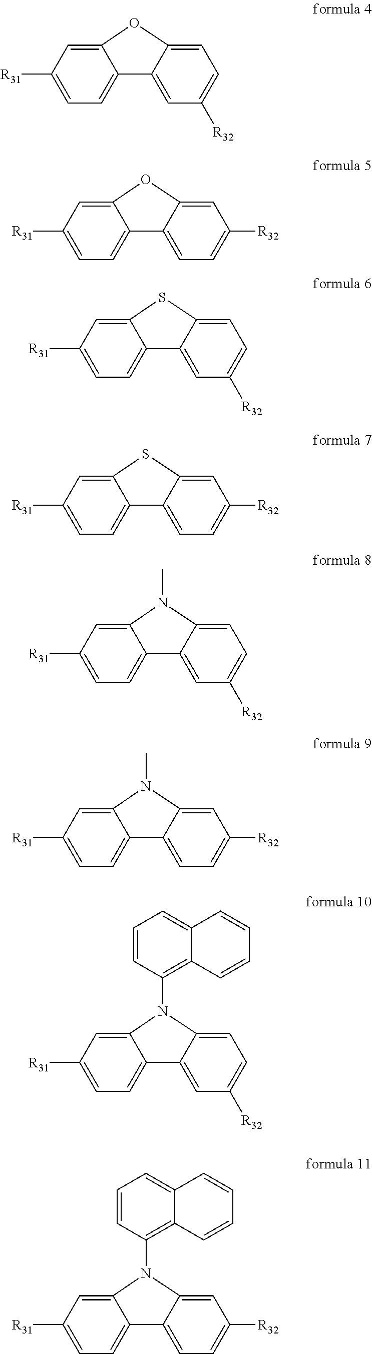

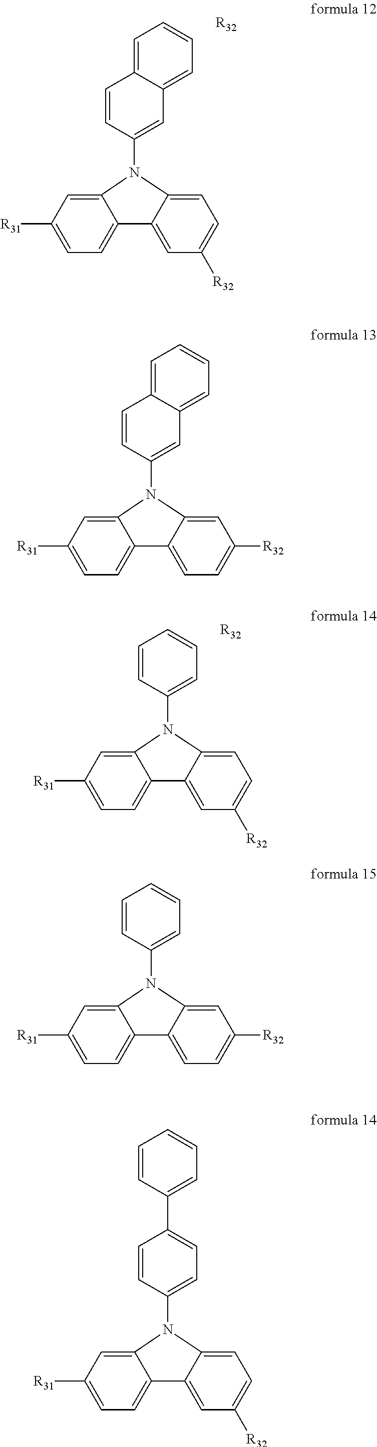

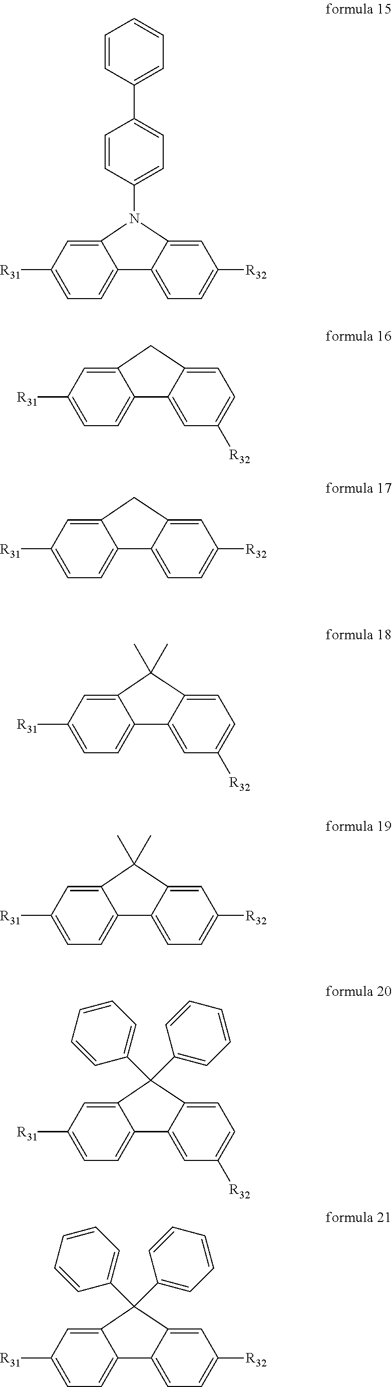

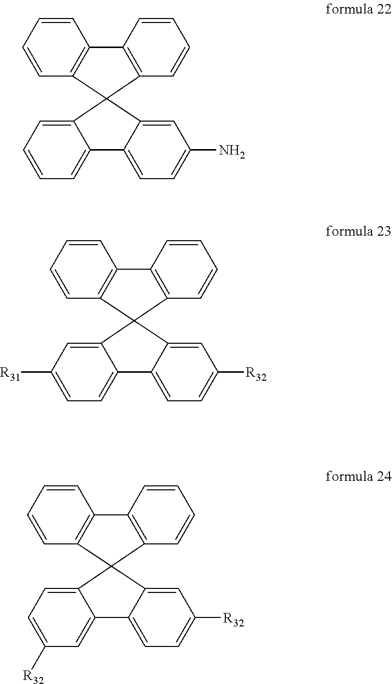

R31 may be hydrogen, phenyl, biphenyl or pyridyl.

R32 may either be, as described above, halogen, a primary amine group NH₂ or a trifluoromethylsulphonic acid radical, depending on whether the radical of the formula 2 or 3 is used as A or B.

R32, however, may also be a spacer which is arranged between A or B and X or NH₂. Examples of suitable spacers include 1,4-phenyl, 1,4-(6-methyl)phenyl, 1,4-(5-methyl)phenyl, 4,4′-biphenyl, 2,6-naphthyl or 1,4-naphthyl. These are, in particular,

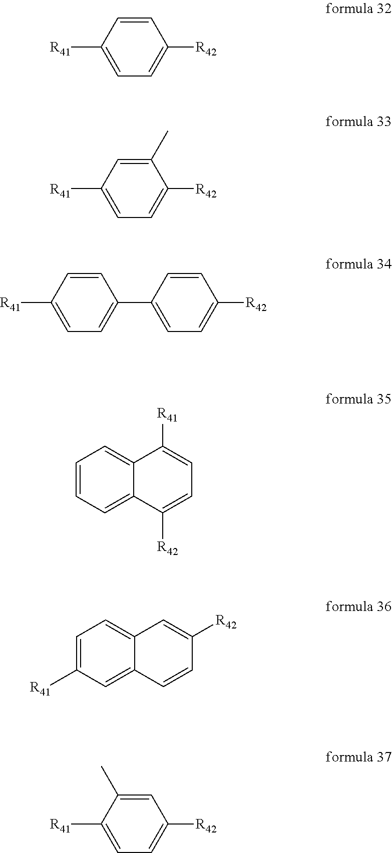

R41 here is a structure of the formulae 2 to 22, and R42 may be halogen as fluorine, chlorine, bromine, iodine or astatine, a primary amine group NH₂ or a trifluoromethylsulphonic acid radical.

In one specific embodiment of the invention, the following compounds are used: the compound of the formula 131 as amine of the formula A-NH₂, and the compound of the formula 132 as haloaromatic of the formula B—X

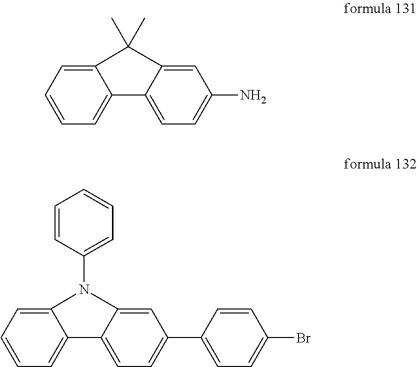 [SMILES: CC1(C)c2ccccc2-c2ccc(N)cc21 ; Brc1ccc(-c2ccc3c4ccccc4n(-c4ccccc4)c3c2)cc1]

The starting compounds, the primary aromatic amine A-NH₂ and the haloaromatic X—B, are used in equimolar proportions. The haloaromatic or the amine may optionally be used as well in an excess of up to 1.1 times or 1.2 times the equimolar ratio.

The palladium complex may be obtained in a manner known in principle. For this purpose, first of all, a palladium compound in a solvent is introduced, and then the desired bis(dialkylphosphinoferrocene) ligand is added, with good results being obtainable with 1,1′-bis(diisopropylphosphino)ferrocene, 1,1′-bis(diisobutylphosphino)ferrocene and 1,1′-bis(di-tert-butylphos-phino)ferrocene. This may be followed by stirring for between 30 and 1000 minutes, more particularly 40 to 400 minutes or 50 to 120 minutes. The reaction temperature may be from about 10° C. to 100° C., more particularly 15° C. to 50° C. or 20° C. to 30° C.

Good results can be obtained simply by stirring at room temperature for about an hour.

The upper temperature limit is dependent essentially on the boiling temperature of the solvent, meaning that high-boiling solvents are required for relatively high reaction temperatures, and the upper temperature limits indicated above may be viewable not as rigid but rather as dependent on the boiling temperature of the solvent.

Suitable solvents are generally aprotic solvents, such as ethers or aromatic solvents. Suitable examples therefore include diethyl ether, tert-butyl ethyl ether, tert-butyl methyl ether, tetrahydrofuran or dioxane, although benzene, toluene or xylene, or else acetonitrile, may also be used.

Good results are achievable through use of very largely anhydrous and oxygen-free solvents, which can be obtained by subjecting the solvents to customary drying techniques.

Palladium compounds suitable as reactants for the palladium complex may be Pd(0) and Pd(II) complexes such as, for example, allylchloro[1,3-bis(2,4,6-trimethylphenyl)imidazol-2-ylidene]palladium(II), (ethylenediamine)palladium(II) chloride, palladium(II) acetate, palladium(II) chloride, palladium pivalate, palladium(II) acetylacetonate, bis(benzonitrile)palladium(II) chloride, bis(acetonitrile)dichloropalladium(II), diamminedichloropalladium(II), dichloro(1,5-cyclooctadiene)palladium(II), palladium(II) nitrate, palladium(II) oxide, palladium(II) oxide hydrate, H₂[PdCl₄], diamminedinitritopalladium(II), palladium(II) sulphates, tetraamminepalladium(II) sulphate [Pd(NH₃)₄]SO₄, tetraamminepalladium(II) hydrogencarbonate, tetraamminepalladium(II) chloride [Pd(NH₃)₄]Cl₂, potassium tetrachloropalladate(II) K₂[PdCl₄], sodium tetrachloropalladate(II) Na₂[PdCl₄], ammonium tetrachloropalladate(II) (NH₄)₂[PdCl₄], tetraamminepalladium(II) nitrate, 1,3-divinyl-1,1,3,3-tetramethyldisiloxanepalladium(0) (also as Pd-VTS, Pd-VTS or palladium-VTS), bis(dibenzylideneacetone)palladium(0) (Pd(dba)₂), (Pd₂(dba)₃), (Pd₂(dba)₃).LM, where LM is a solvent, more particularly CHCl₃ or CH₂Cl₂. In this way a catalyst solution is obtained.

Following the preparation of the palladium complex, the procedure may continue by the addition to the catalyst solution, in a one-pot reaction, of the further reactants: that is, the primary aromatic amine A-NH₂, the haloaromatic X—B and the base that is required for the Hartwig-Buchwald coupling. Suitable bases are alkali metal and alkaline earth metal hydroxides, such as lithium hydroxide, potassium hydroxide or sodium hydroxide, for example. Also suitable are tertiary organic amines or alcoholates such as tributylamine, triethylamine, alkali metal alcoholates such as lithium ethanolate, sodium ethanolate or potassium ethanolate, lithium tert-butanolate, sodium tert-butanolate or potassium tert-butanolate, lithium bis(trimethylsilyl)amide, sodium bis(trimethylsilyl)amide, potassium bis(trimethylsilyl)amide, individually or in combination with one another, as base.

Relative to the total molar amount of the two reactants, the primary aromatic amine A-NH2 and the haloaromatic X—B, the base may be used in general in amounts of 40% to 80%, more particularly 50% to 70% or 55% to 65%. This means that if, for example, 20 mmol each of the primary aromatic amine A-NH₂ and of the haloaromatic X—B are used, in total therefore mmol, the base may be used in amounts of 16 mmol to 32 mmol, more particularly 20 mmol to 28 mmol or from 22 mmol to 26 mmol.

Alternatively to this procedure, the primary aromatic amine A-NH₂, the haloaromatic X—B and the base may also be introduced with addition of a solvent, and the catalyst solution metered in in appropriate amount.

Suitable solvents for the coupling reaction between the aromatic amine A-NH₂ and the haloaromatic X—B are the customary solvents for Hartwig-Buchwald couplings, in other words alcohols such as ethanol, propanol, isopropanol, butanol, isobutanol, but also, for example, ethylene glycol, ethers such as diethyl ether, tert-butyl ethyl ether, tert-butyl methyl ether, tetrahydrofuran, dioxane, ethylene glycol dimethyl ether, bis(2-methoxyethyl) ether, aromatic solvents such as benzene, toluene or xylenes, such as o-xylene, p-xylene, m-xylene and mixtures thereof, or combinations of these solvents. With the solvents, care should be taken to ensure that they are inert under the conditions of Hartwig-Buchwald coupling, since amino groups or halogen groups may disrupt the desired reaction.

The amount of catalyst needed for the conversion without the double arylation that occurs as a secondary reaction, forming the tertiary amine, is between 0.01% to 1.5 mol %, or from 0.1 to 1 mol %, or from 0.3 mol % to 0.8 mol %. The figure in mol % is based on the total amount in mol of the two reactants, the primary aromatic amine A-NH₂ and the haloaromatic X—B.

The conversion rates of the reactants are at least 90%, more particularly at least 95% or at least 97%. The double arylation which often occurs as a secondary reaction, forming the tertiary amine, is almost completely suppressed here.

The reaction temperatures are generally 5° C. to 150° C., or 20° C. to 140° C., or 30° C. to 130° C., more particularly 60° C. to 120° C. or 70° C. to 111° C. The reaction times are in general one hour to 36 hours, or 4 hours to 24 hours or 6 hours to 16 hours or 8 hours to 12 hours.

Added as catalyst in one advantageous embodiment, in addition to the palladium complex where the palladium atom is complexed with a bis(dialkylphosphinoferrocene) ligand of the formula 1, is the corresponding ligand, in other words the bis(dialkylphosphinoferrocene) ligand that is used in the palladium complex. Having emerged as being sensible here is an addition in a ratio of palladium complex to bis(dialkylphosphinoferrocene) ligand in the range from 1:10 to 10:1, or from 1:5 to 5:1, more particularly from 2.5:1 to 1:2.5, such as, for example, 2:1. The ratio is based on the molar amounts of palladium complex and bis(dialkylphosphinoferrocene) ligand. This means for example that when using 0.2 mol % of palladium complex, 0.1 mol % of bis(dialkylphosphinoferrocene) ligand is added.

With the procedure described above, the additional amount of bis(dialkyl-phosphinoferrocene) ligand may be added, for example, to the catalyst solution, either before, during or after its preparation from the starting products, and this solution may then be stored until the reaction is carried out.

Alternatively, the bis(dialkylphosphinoferrocene) ligand, the primary aromatic amine A-NH₂, the haloaromatic X—B and the base may be introduced together, with addition of a solvent, and the catalyst solution may be metered in in appropriate amount.

A further alternative is to prepare a solid catalyst from the catalyst solution. For this purpose, the catalyst solution is admixed with an additive which forms a palladium complex which is of low solubility and crystallizes well. A stoichiometric compound is precipitated here. Under the conditions of the precipitation, in other words with a sufficiently low solvent volume, this compound is of sufficiently low solubility to precipitate, but soluble enough to be used again to prepare a catalyst solution.

In the preparation of the palladium complex, the palladium atom is complexed not only by the bis(dialkylphosphinoferrocene) ligand but also by ligands from the palladium compound used in preparing the palladium complex—in other words, for example, by 1,3-divinyl-1,1,3,3-tetramethyl-disiloxane (also known as VTS or VS), bis(dibenzylideneacetone) (dba) or others.

These palladium complexes, however, are readily soluble. The function of the additive is to complex the palladium atom and thereby to form a relatively poorly soluble palladium complex, which can easily be separated off and processed further. Examples of suitable additives include naphthoquinone, maleic anhydride, maleimide, diethyl maleate or norbomene, more particularly naphthoquinone or maleimide. Low-solubility, readily crystallizing palladium complexes are then formed, which can be isolated by filtration, washed and dried. A stoichiometric compound is precipitated here. Under the conditions of the precipitation, in other words with a sufficiently low solvent volume, this compound is of sufficiently low solubility to precipitate, but soluble enough to be used again to prepare a catalyst solution.

The present patent application therefore also relates to the compounds 1,1′-bis(diisopropylphosphino)ferrocenepalladium(0)-maleimide [Pd(dippf)(maleimide)], 1,1′-bis(diiso-propylphosphino)ferrocenepalladium(0)-1,3-divinyl-1,1,3,3-tetramethyldisiloxane [Pd(dippf)(VTS)], 1,1′-bis(dlisopropylphosphino)ferrocenepalladium(0)-bis(dibenzylideneacetone) [Pd(dippf)(dba)], 1,1′-bis(diisopropylphosphino)ferrocenepalladium(0)-naphthoquinone [Pd(dippf)(naphthoquinone)], 1,1′-bis(diisopropylphosphino)ferrocenepalladium(0)-maleic anhydride [Pd(dippf)(maleic anhydride)], 1,1′-bis(diisopropyl-phosphino)ferrocenepalladium(0)-diethyl maleate [Pd(dippf)(diethyl maleate)], and 1,1′-bis(dilsopropylphosphino)ferrocenepalladium(0)-norbornene [Pd(dippf)(norbomene)].

The palladium atom (palladium in oxidation state 0) is often triply coordinated and the complexes are generally trigonal-planar, meaning that compounds such as 1,3-divinyl-1,1,3,3-tetramethyldisiloxane (VTS or VS) or bis(dibenzylideneacetone), which contain more than one double bond, complex the palladium atom only with one of their double bonds. The second double bond complexes either nothing or a further palladium atom.

The structure of some of these compounds may be represented as follows:

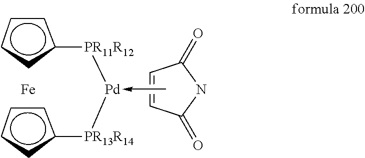

The radicals R11 to R14 in formula 200 may be identical or different, as described above, and are, in particular, alkyl radicals having one to five carbon atoms. If R11 to R14 are isopropyl radicals, formula 200 shows 1,1′-bis(diisopropylphosphino)ferrocenepalladium(0)-maleimide [Pd(dippf)(maleimide)].

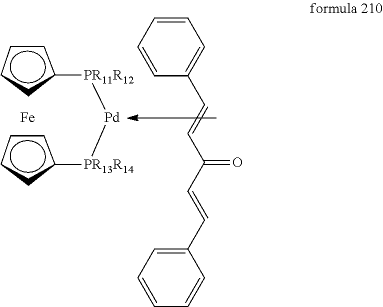

The radicals R11 to R14 In formula 210 may be identical or different, as described above, and are, in particular, alkyl radicals having one to five carbon atoms. If R11 to R14 are isopropyl radicals, formula 210 shows 1,1′-bis(diisopropylphosphino)ferrocenepalladium(0)-bis(dibenzylideneacetone) [Pd(dippf)(dba)].

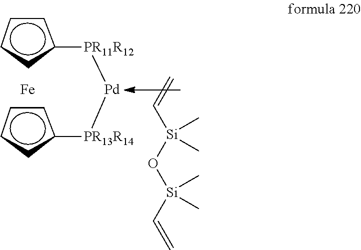

The radicals R11 to R14 in formula 220 may be identical or different, as described above, and are, in particular, alkyl radicals having one to five carbon atoms. If R11 to R14 are isopropyl radicals, formula 220 shows 1,1′-bis(diisopropylphosphino)ferrocenepalladium(0)-1,3-divinyl-1,1,3,3-tetramethyldisiloxane [Pd(dippf)(VTS)].

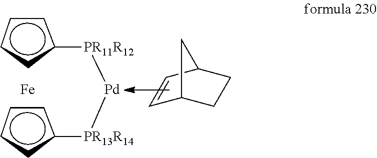

The radicals R11 to R14 in formula 230 may be identical or different, as described above, and are, in particular, alkyl radicals having one to five carbon atoms. If R11 to R14 are isopropyl radicals, formula 230 shows 1,1′-bis(diisopropylphosphino)ferrocenepalladium(0)-norbornene [Pd(dippf)(norbornene)].

These solid palladium complexes may likewise be used, as described above, as catalyst, in which case they are introduced initially or else added as a solid or solution to the reactants and the base.

In one advantageous embodiment, besides the solid palladium complex, the corresponding ligand is added as well, in other words the bis(dialkylphosphinoferrocene) ligand used in the solid palladium complex.

Having emerged as being sensible here is an addition in a ratio of solid palladium complex to bis(dialkylphosphinoferrocene) ligand in the range from 1:10 to 10:1, or from 1:5 to 5:1, more particularly from 2.5:1 to 1:2.5, such as, for example, 2:1. The ratio is based on the molar amounts of solid palladium complex and bis(dialkylphosphinoferrocene) ligand. This means for example that when using 0.2 mol % of solid complex, 0.1 mol % of bis(dialkylphosphinoferrocene) ligand is added. Alternatively, the solid palladium complex may be mixed with additional bis(dialkylphosphino-ferrocene) ligand in the solid state to give a powder mixture. This powder mixture is stable on storage and is easy to work with.

### Examples

### Example 1

### Preparation of the Catalyst Solution

1,1′-Bis(dilsopropylphosphino)ferrocene (dippf) (213 mg, 0.50 mmol) was dissolved in diethyl ether (5 mL) and 1,3-divinyl-1,1,3,3-tetramethyldisiloxanepalladium(0) (Pd-VTS) in 2,4,6,8-tetramethylcyclotetrasiloxane (0.50 mL, 0.50 mmol Pd) was added dropwise. The orange mixture was stirred at room temperature for 1 hour.

### Example 2

### Preparation of the Solid Catalyst [Pd(Dippf)(Maleimide)]

The procedure was initially as in example 1. Then maleimide (99.1 mg, 1.00 mmol) in diethyl ether (5 mL) was added and the mixture was treated in an ultrasound bath for ten minutes, whereupon a yellow precipitate formed. After the solid had settled, the supernatant solution was removed and the residue was washed with diethyl ether (3×5 mL). Drying under reduced pressure (10⁻² mbar) gave the desired palladium complex, isolated as a yellow solid (291 mg, 0.47 mmol, 94%).

Analytical Data:

¹H NMR (400 MHz, dioxane-d₈): δ=7.84 (s, 1H, N—H), 4.44-4.39 (m, 6H, ferrocene-H), 4.37-4.33 (m, 2H, ferrocene-H), 2.53-2.40 (m, 2H), 2.35-2.20 (m, 2H), 1.34 (d, 3=7.0 Hz, 3H), 1.30 (d, 3=7.0 Hz, 3H), 1.26 (d, J=7.3 Hz, 3H), 1.22 (d, 3=7.3 Hz, 6H), 1.18 (d, 3=7.3 Hz, 3H), 1.13 (d, 3=7.3 Hz, 3H), 1.09 ppm (d, 3=7.3 Hz, 3H). ³¹P NMR (162 MHz, dioxane-d₈): δ=: 38.87 ppm (s, 2P).

### Example 3

### Syntheses on the Preparative Scale

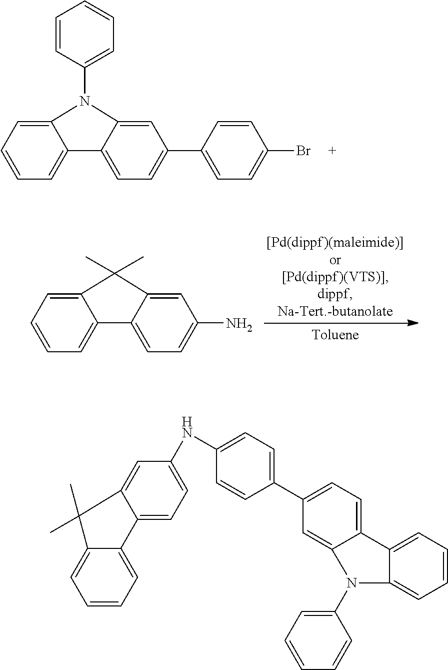 [SMILES: Brc1ccc(-c2ccc3c4ccccc4n(-c4ccccc4)c3c2)cc1 ; CC1(C)c2ccccc2-c2ccc(N)cc21 ; CC1(C)c2ccccc2-c2ccc(Nc3ccc(-c4ccc5c6ccccc6n(-c6ccccc6)c5c4)cc3)cc21]

### Diagram 1

### Example 3a

### Use of [Pd(Dippf)(Maleimide)] as Palladium Complex

A dry three-necked flask with reflux condenser, gas inlet, excess-pressure valve and magnetic stirring rod was charged with the aryl bromide 132 (7.97 g, 20.0 mmol), the amine 131 (4.17 g, 20.0 mmol), 1,1′-bis(diisopropylphosphino)ferrocene (8.5 mg, 0.02 mmol) and sodium tert-butanolate (2.35 g, 24.0 mmol) and was brought under a nitrogen atmosphere by multiple evacuation and gas admission. Toluene (30 mL) and [Pd(dippf)(maleimide)](24.9 mg, 0.04 mmol, from example 2) in toluene (10 mL) were added and the mixture was heated at 70° C. for 20 hours. The reaction was monitored by thin-layer chromatography and it was found that after this time full conversion had been reached. After cooling had taken place to room temperature, water (60 mL) was added and the reaction mixture was extracted with dichloromethane (150 mL). The organic phase was separated off, dried over magnesium sulphate (5 g) and filtered through basic aluminium oxide (10 g). The solvent was removed under reduced pressure (40° C., 10 mbar) and the slightly yellow residue was washed with diethyl ether (3×20 mL). Drying under reduced pressure (2 h, 10⁻² mbar) gave the product as a colourless solid (10.2 g, 19.4 mmol, 97%).

¹H NMR (400 MHz, chloroform-d): δ=8.41 (d, J=1.3 Hz, 1H), 8.26 (d, J=7.8 Hz, 1H), 7.75-7.60 (m, 9H), 7.55-7.44 (m, 5H), 7.41-7.21 (m, 6H), 7.13 (d, J=7.0 Hz, 1H), 5.91 (s, 1H), 1.55 ppm (s, 6H).

¹³C NMR (101 MHz, chloroform-d): δ=155.3, 153.1, 142.6, 142.0, 141.3, 140.0, 139.3, 137.7, 134.6, 133.2, 132.5, 129.9 (2C), 128.1, 127.4 (2C), 127.0 (2C), 126.9, 126.0 (2C), 125.0, 123.9, 123.5 (2C), 122.4, 120.8, 120.3, 120.0, 119.9, 119.1, 118.1, 116.8, 112.2, 110.0, 109.9, 46.8, 27.2 ppm (2C).

Elemental analysis: Calculated for C₃H₃₀N₂: C, 88.94; H, 5.74; N, 5.32. found: C, 88.64; H, 5.91; N, 5.22.

### Example 3b

### Use of [Pd(Dippf)(VTS)] as Palladium Complex

For this reaction, a catalyst stock solution was used, prepared, similarly to the description in example 1, from 1,1′-bis(diisopropylphosphino)ferrocene (213.8 mg, 0.50 mmol), toluene (0.5 mL) and a mixture of 1,3-divinyl-1,1,3,3-tetramethyldisiloxanepalladium(0) (Pd-VTS) in 2,4,6,8-tetramethylcyclotetrasiloxane (0.5 mL, 10.87% palladium). The mixture was stirred at room temperature for an hour.

A dry three-necked flask with reflux condenser, gas inlet, excess-pressure valve and magnetic stirring rod was charged with the aryl bromide 132 (7.97 g, 20.0 mmol), the amine 131 (4.17 g, 20.0 mmol) and sodium tert-butanolate (2.35 g, 24.0 mmol) and was brought under a nitrogen atmosphere by evacuation and gas admission. Toluene (40 mL) and the catalyst stock solution (100 μL, 91.8 mg, 0.04 mmol of palladium) were added and the mixture was heated at 70° C. for 20 hours. The reaction was monitored by thin-layer chromatography and it was found that after this time full conversion had been reached. After cooling had taken place to room temperature, water (60 mL) was added and the reaction mixture was extracted with dichloromethane (150 mL). The organic phase was separated off, dried over magnesium sulphate (5 g) and filtered through basic aluminium oxide (10 g). The solvent was removed under reduced pressure (40° C., 10 mbar) and the slightly yellow residue was washed with ether (3×20 mL). Drying under reduced pressure (2 h, 10⁻² mbar) gave the product as a colourless solid (9.71 g, 18.4 mmol, 92%).

The reaction equation is shown in diagram 1. Multiple arylation of the primary amine 131 by the aryl bromide 132 was not observed in any of the examples.

### Examples 4a to 4i

### General Protocol

All experiments were carried out in 20 mL headspace vials for gas chromatography that were sealed with crimped aluminium caps featuring Teflon-coated butyl rubber septa (both available, for example, from VWR). For the heating of the vessels, cylindrical aluminium blocks 8 cm high were used, corresponding precisely in their diameter to the hotplates of laboratory magnetic stirrers (e.g. Heidolph Mr 2002). These aluminium blocks were provided with ten holes 7 cm deep and the same diameter as the reaction vessels, and a hole for accommodating a temperature sensor.

Vacuum distributors for connection to the Schlenk line were made for the simultaneous evacuation and refilling of ten vessels at a time. For this purpose, ten vacuum-compatible 3 mm Teflon tubes were each connected at one end with adapters to accommodate Luer-Lock syringe needles and were connected at the other end to a steel tube, which can be connected via a vacuum hose to the Schlenk line.

For the implementation of the experiments, the aryl bromide (1.00 mmol), the corresponding primary amine (1.00 mmol) and sodium tert-butanolate (118 mg, 1.20 mmol) were weighed out in air into the reaction vessels, 20 mm magnetic stirring cores were added, and the vessels were given an airtight seal with septum caps, using crimping tongs. Ten reaction vessels at a time were introduced into the holes in an aluminium block, and connected to the vacuum distributor via hollow needles which were inserted through the septum caps.

The reaction vessels were subsequently evacuated and charged with nitrogen gas three times in succession, in unison. When the reaction vessels had been given an inert gas atmosphere in this way, pressure compensation with the external atmosphere was produced at the vacuum line via an excess-pressure valve. Using a syringe, a stock solution of [Pd(dippf)(maleimide)](1.24 mg, 0.002 mmol) and dippf (0.85 mg, 0.002 mmol) in toluene (2 mL) was injected through the septum caps. The aluminium block was then brought to 70° C. and the needles of the vacuum distributor were removed. After a reaction time of 20 hours, the vessels were cooled and carefully opened and the reaction medium was diluted with dichloromethane (30 mL) and water (30 mL). The aqueous phase was adjusted to a pH of 7 using 1N hydrochloric acid, separated from the organic phase and extracted with dichloromethane (2×20 mL). The combined organic phases were dried over magnesium sulphate, filtered and finally analyzed by thin-layer chromatography.

The solvent was removed under reduced pressure (40° C., 500 mbar) and the crude product which remained was purified by column chromatography (basic Al₂O₃, diethyl ether:hexane or ethyl acetate:hexane).

### Example 4a

### Synthesis of Compound 3a

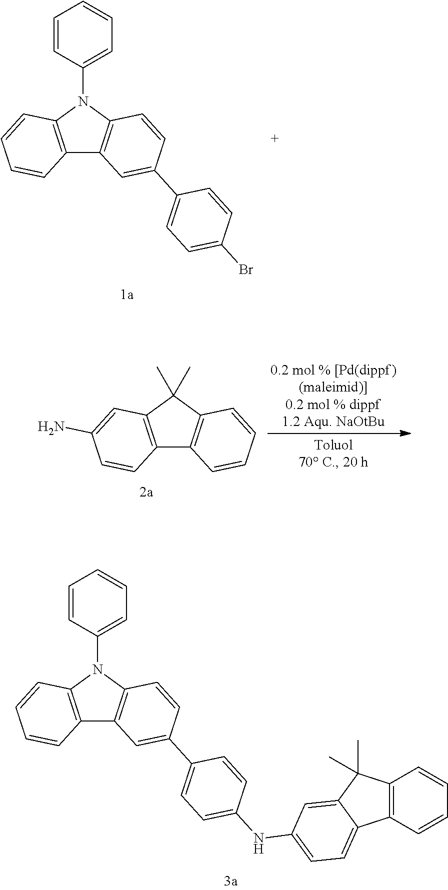 [SMILES: Brc1ccc(-c2ccc3c(c2)c2ccccc2n3-c2ccccc2)cc1 ; CC1(C)c2ccccc2-c2ccc(N)cc21 ; CC1(C)c2ccccc2-c2ccc(Nc3ccc(-c4ccc5c(c4)c4ccccc4n5-c4ccccc4)cc3)cc21]

According to the general protocol, compound 3a was prepared starting from 1a (398 mg, 1.00 mmol) and 2a (209 mg, 1.00 mmol) and was isolated by column chromatography (Al₂O₃, diethyl ether:hexane=1:1) in 94% yield (496 mg, 0.94 mmol). ¹H NMR (400 MHz, chloroform-d): δ=8.41 (d, J=1.3 Hz, 1H), 8.26 (d, J=7.8 Hz, 1H), 7.75-7.60 (m, 9H), 7.55-7.44 (m, 5H), 7.41-7.21 (m, 6H), 7.13 (d, J=7.0 Hz, 1H), 5.91 (s, 1H), 1.55 ppm (s, 6H), ¹³C NMR (101 MHz, chloroform-d): δ=155.3, 153.1, 142.6, 142.0, 141.3, 140.0, 139.3, 137.7, 134.6, 133.2, 132.5, 129.9 (2C), 128.1, 127.4 (2C), 127.0 (2C), 126.9, 126.0 (2C), 125.0, 123.9, 123.5 (2C), 122.4, 120.8, 120.3, 120.0, 119.9, 119.1, 118.1, 116.8, 112.2, 110.0, 109.9, 46.8, 27.2 ppm (2C). CHN: Calculated for C₃H₃₀N₂: C, 88.94; H, 5.74; N, 5.32. found: C, 88.79; H, 5.86; N, 5.19.

### Example 4b

### Synthesis of Compound 3b

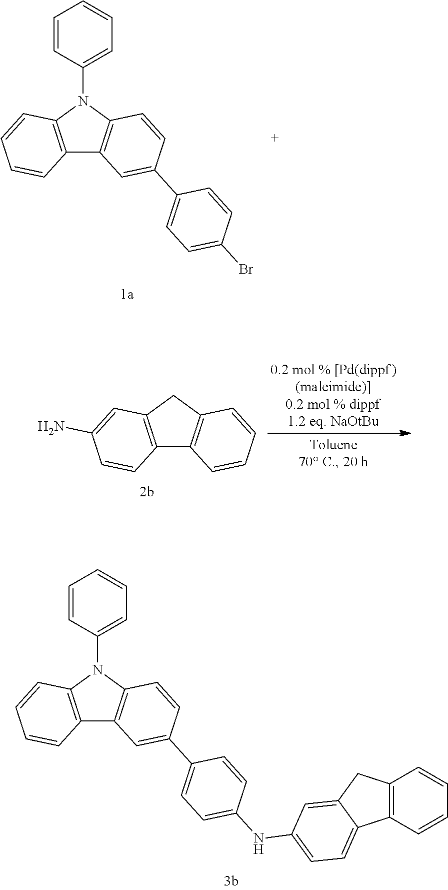 [SMILES: Brc1ccc(-c2ccc3c(c2)c2ccccc2n3-c2ccccc2)cc1 ; Nc1ccc2c(c1)Cc1ccccc1-2 ; c1ccc(-n2c3ccccc3c3cc(-c4ccc(Nc5ccc6c(c5)Cc5ccccc5-6)cc4)ccc32)cc1]

According to the general protocol, compound 3b was prepared starting from 1a (398 mg, 1.00 mmol) and 2b (181 mg, 1.00 mmol) and was isolated by column chromatography (Al₂O₃, diethyl ether:hexane=1:1) in 92% yield (460 mg, 0.92 mmol). ¹H NMR (400 MHz, DMSO-d₆): δ=8.54 (d, J=1.5 Hz, 1H), 8.44 (s, 1H), 8.35 (d, J=7.8 Hz, 1H), 7.77-□7.63 (m, 9H), 7.55 (m, 1H), 7.51 (d, J=7.5 Hz, 1H), 7.47-□7.36 (m, 4H), 7.31 (m, 2H), 7.26 (d, J=8.8 Hz, 2H), 7.20 (dt, J=7.5 Hz, 1.3 Hz, 1H), 7.15 (dd, J=8.3 Hz, 2.0 Hz, 1H), 3.87 ppm (s, 2H), ¹³C NMR (101 MHz, DMSO-d₆): δ=144.6, 142.7, 142.3, 142.2, 141.5, 140.5, 139.1, 136.9, 133.3, 132.6, 132.2, 130.2 (2C), 127.6, 127.5 (2C), 126.7, 126.6 (2C), 126.4, 125.3, 124.9, 124.7, 123.4, 123.0, 120.8, 120.7, 120.1, 118.8, 117.6, 117.4 (2C), 117.3, 115.9, 113.1, 110.0, 109.7 ppm. CHN: Calculated for C₃₇H₂₆N₂: C, 89.13; H, 5.26; N, 5.62. found: C, 88.83; H, 5.25; N, 5.54.

### Example 4c

### Synthesis of Compound 5c

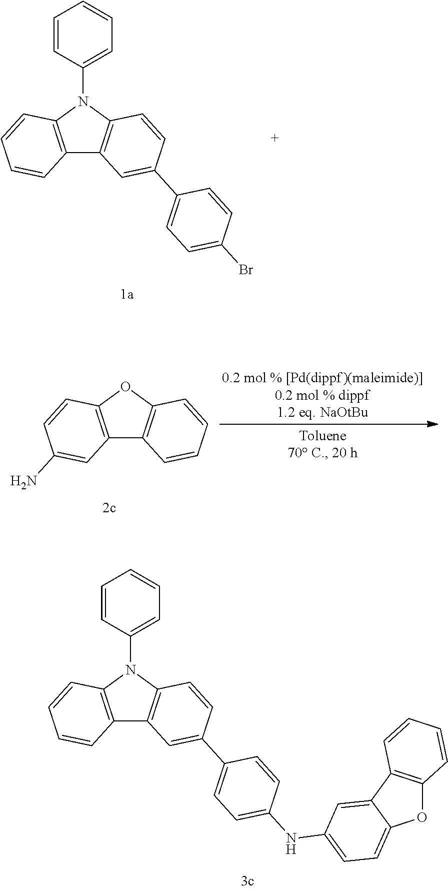 [SMILES: Brc1ccc(-c2ccc3c(c2)c2ccccc2n3-c2ccccc2)cc1 ; Nc1ccc2oc3ccccc3c2c1 ; c1ccc(-n2c3ccccc3c3cc(-c4ccc(Nc5ccc6oc7ccccc7c6c5)cc4)ccc32)cc1]

According to the general protocol, compound 3c was prepared starting from 1a (398 mg, 1.00 mmol) and 2c (183 mg, 1.00 mmol) and was isolated by column chromatography (Al₂O₃, diethyl ether:hexane=1:1) in 88% yield (442 mg, 0.88 mmol). ¹H NMR (400 MHz, DMSO-d₆): δ=8.72 (s, 1H), 8.55 (d, J=1.5 Hz, 1H), 8.35 (d, J=7.8 Hz, 1H), 7.99-7.93 (m, 2H), 7.77-7.62 (m, 7H), 7.60 (d, J=8.0 Hz, 1H), 7.54 (m, 1H), 7.47-7.27 (m, 9H), 7.16 ppm (dd, J=8.5 Hz, 1.8 Hz, 1H), ¹³C NMR (101 MHz, DMSO-d₆): δ=157.1, 155.3, 144.0, 141.6, 140.5, 139.2, 136.9, 133.0, 132.5, 130.2 (2C), 127.6, 127.5 (2C), 126.6 (2C), 126.4, 125.6, 124.7, 124.2, 123.4, 123.0 (2C), 121.6, 120.8, 120.1, 119.7, 118.0 (2C), 117.7, 115.5, 113.1, 111.2, 110.0, 109.7, 97.9 ppm. CHN: Calculated for C₃₆H₂₄N₂₀: C, 86.38; H, 4.83; N, 5.60. found: C, 86.02; H, 5.04; N, 5.43.

### Example 4c

### Synthesis of Compound 3d

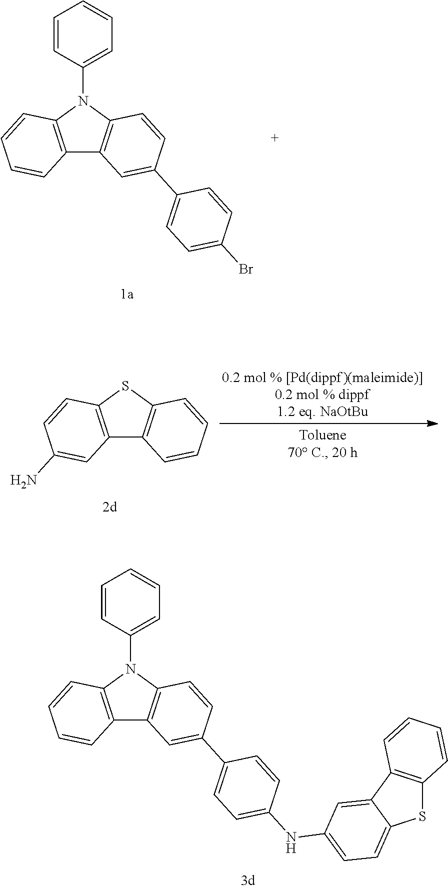 [SMILES: Brc1ccc(-c2ccc3c(c2)c2ccccc2n3-c2ccccc2)cc1 ; Nc1ccc2sc3ccccc3c2c1 ; c1ccc(-n2c3ccccc3c3cc(-c4ccc(Nc5ccc6sc7ccccc7c6c5)cc4)ccc32)cc1]

According to the general protocol, compound 3d was prepared starting from 1a (398 mg, 1.00 mmol) and 2d (199 mg, 1.00 mmol) and was isolated by column chromatography (Al₂O₃, diethyl ether:hexane=1:1) in 96% yield (496 mg, 0.96 mmol). ¹H NMR (400 MHz, DMSO-d₆): δ=8.54 (d, J=1.5 Hz, 1H), 8.51 (s, 1H), 8.35 (d, J=7.5 Hz, 1H), 8.25 (m, 1H), 8.07 (d, J=2.0 Hz, 1H), 7.99 (m, 1H), 7.89 (d, J=8.5 Hz, 1H), 7.75-7.63 (m, 7H), 7.55 (m, 1H), 7.48 (m, 2H), 7.45-7.37 (3H), 7.36-7.27 ppm (m, 4H), ¹³C NMR (101 MHz, DMSO-d₆): δ=142.7, 141.0, 140.5, 139.5, 139.1, 136.9, 136.0, 134.9, 132.6, 132.1, 130.2 (2C), 129.7, 127.6, 127.5 (2C), 126.9, 126.6 (2C), 126.4, 124.7, 124.5, 123.6, 123.4, 123.1, 123.0, 121.9, 120.8, 120.1, 119.0, 117.6, 116.8 (2C), 110.0, 109.7, 109.2 ppm. CHN: Calculated for C₃₆H₂₄N₂S: C, 83.69; H, 4.68; N, 5.42; S, 6.21. found: C, 83.40; H, 4.76; N, 5.35; S, 6.31.

### Example 4d

### Synthesis of Compound 3e

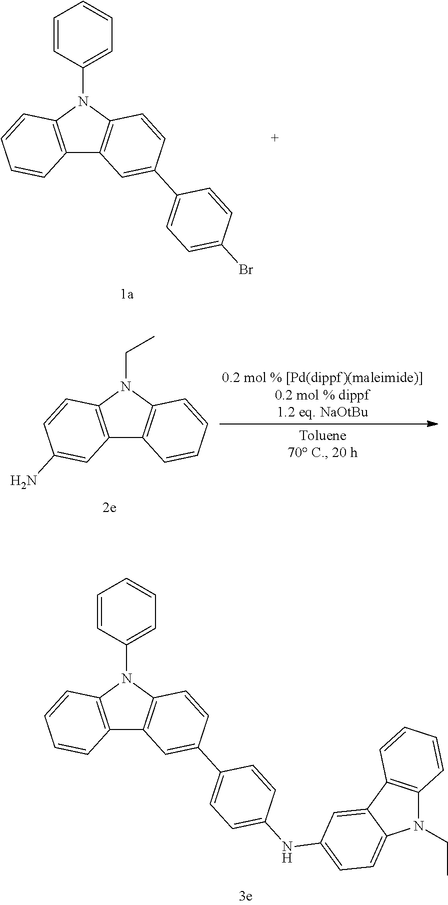 [SMILES: Brc1ccc(-c2ccc3c(c2)c2ccccc2n3-c2ccccc2)cc1 ; CCn1c2ccccc2c2cc(N)ccc21 ; CCn1c2ccccc2c2cc(Nc3ccc(-c4ccc5c(c4)c4ccccc4n5-c4ccccc4)cc3)ccc21]

According to the general protocol, compound 3e was prepared starting from 1a (398 mg, 1.00 mmol) and 2e (221 mg, 1.00 mmol) and was isolated by column chromatography (Al₂O₃, ethyl acetate:hexane=1:2) in 95% yield (502 mg, 0.95 mmol). ¹H NMR (400 MHz, DMSO-d₆): δ=8.50 (d, J=1.5 Hz, 1H), 8.33 (d, J=7.8 Hz, 1H), 8.14 (s, 1H), 8.11 (d, J=7.5 Hz, 1H), 7.96 (d, J=2.0 Hz, 1H), 7.72-7.61 (m, 7H), 7.58-7.51 (m, 3H), 7.46-7.37 (m, 4H), 7.35-7.27 (m, 2H), 7.17-7.11 (m, 3H), 4.41 (q, J=7.0 Hz, 2H), 1.31 ppm (t, J=7.0 Hz, 3H), ¹³C NMR (101 MHz, DMSO-d₆): δ=144.6, 142.7, 142.3, 142.2, 141.5, 140.5, 139.1, 136.9, 133.3, 132.6, 132.2, 130.2 (2C), 127.6, 127.5 (2C), 126.7, 126.6 (2C), 126.4, 125.3, 124.9, 124.7, 123.4, 123.0, 120.8, 120.7, 120.1, 118.8, 117.6, 117.4 (2C), 117.3, 115.9, 113.1, 110.0, 109.7, 36.5 ppm. CHN: Calculated for C₃₈H₂₉N₃: C, 86.50; H, 5.54; N, 7.96. found: C, 86.32; H, 5.63; N, 7.90.

### Example 4e

### Synthesis of Compound 3f

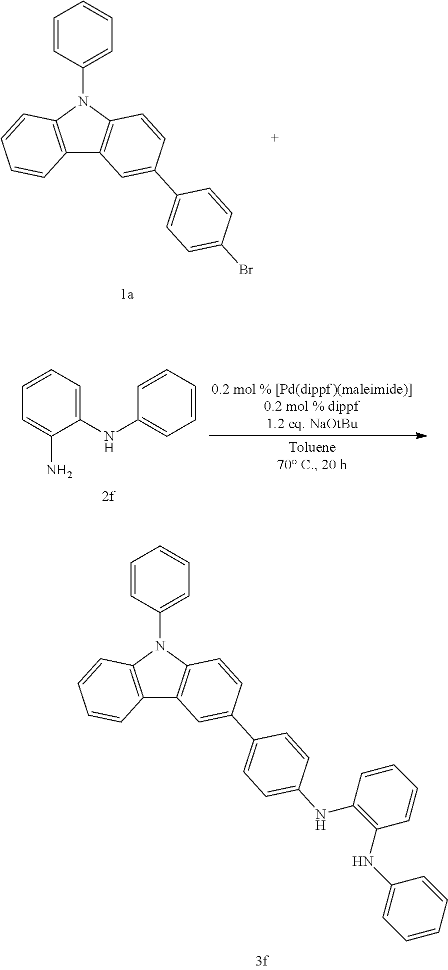 [SMILES: Brc1ccc(-c2ccc3c(c2)c2ccccc2n3-c2ccccc2)cc1 ; Nc1ccccc1Nc1ccccc1 ; c1ccc(Nc2ccccc2Nc2ccc(-c3ccc4c(c3)c3ccccc3n4-c3ccccc3)cc2)cc1]

According to the general protocol, compound 3f was prepared starting from 1a (398 mg, 1.00 mmol) and 2f (188 mg, 1.00 mmol) and was isolated by column chromatography (Al₂O₃, ethyl acetate:hexane=1:2) in 84% yield (423 mg, 0.84 mmol). ¹H NMR (400 MHz, DMSO-d₆): 5=8.49 (d, 3=1.5 Hz, 1H), 8.33 (d, 3=7.8 Hz, 1H), 7.72-7.61 (m, 7H), 7.53 (m, 1H), 7.46-7.37 (m, 4H), 7.36 (s, 1H), 7.34-7.26 (m, 3H), 7.19 (m, 2H), 7.10 (d, J=8.5 Hz, 2H), 6.99 (dd, J=8.7 Hz, 0.9 Hz, 2H), 6.94 (m, 2H), 6.76 ppm (m, 1H), ¹³C NMR (101 MHz, DMSO-d₆): δ=144.5, 143.4, 140.5, 139.0, 136.9, 134.6 (2C), 132.7, 131.6, 130.2 (2C), 129.0 (2C), 127.6, 127.3 (2C), 126.6 (2C), 126.3, 124.6, 123.4, 123.0, 122.1, 122.0, 120.8, 120.1, 119.9 (2C), 119.0, 117.5, 116.7 (2C), 116.2 (2C), 109.9, 109.6 ppm. CHN: Calculated for C₃₆H₂₇N₃: C, 86.20; H, 5.43; N, 8.38. found: C, 85.82; H, 5.62; N, 8.22.

### Example 4f

### Synthesis of Compound 3g

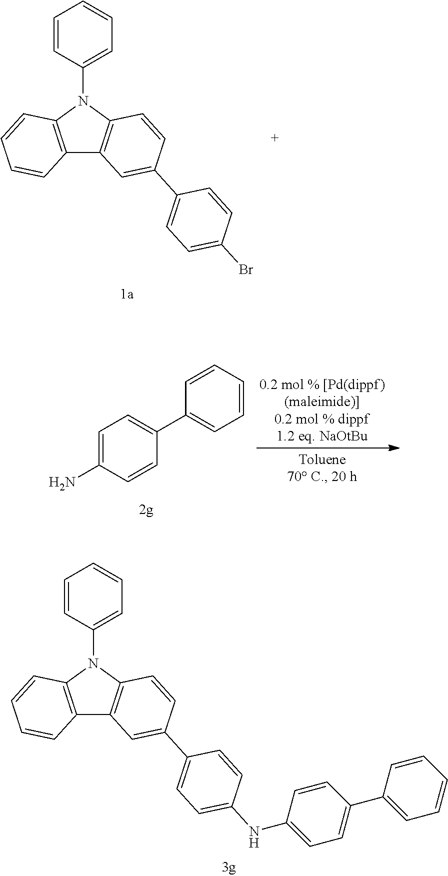 [SMILES: Brc1ccc(-c2ccc3c(c2)c2ccccc2n3-c2ccccc2)cc1 ; Nc1ccc(-c2ccccc2)cc1 ; c1ccc(-c2ccc(Nc3ccc(-c4ccc5c(c4)c4ccccc4n5-c4ccccc4)cc3)cc2)cc1]

According to the general protocol, compound 3g was prepared starting from 1a (398 mg, 1.00 mmol) and 2g (169 mg, 1.00 mmol) and was isolated by column chromatography (Al₂O₃, diethyl ether:hexane=1:1) in 95% yield (462 mg, 0.95 mmol). ¹H NMR (400 MHz, DMSO-d₆): δ=8.53 (d, J=1.3 Hz, 1H), 8.47 (s, 1H), 8.35 (d, J=7.5 Hz, 1H), 7.74-7.53 (m, 12H), 7.47-7.38 (m, 5H), 7.34-7.19 ppm (m, 6H). ¹³C NMR (101 MHz, DMSO-d₆): δ=143.0, 142.9, 141.9, 140.5, 140.1, 139.1, 136.9, 132.6, 132.5, 131.2, 130.2 (2C), 128.9 (2C), 127.6, 127.5 (2C), 127.4 (2C), 126.6 (2C), 126.4 (2C), 125.8 (2C), 124.7, 123.4, 123.0, 120.8, 120.1, 117.7, 117.5 (2C), 116.8, 110.0, 109.7 ppm. CHN: Calculated for C₃H₂₆N₂: C, 88.86; H, 5.39; N, 5.76. found: C, 88.49; H, 5.39; N, 5.68.

### Example 4g

### Synthesis of Compound 3i

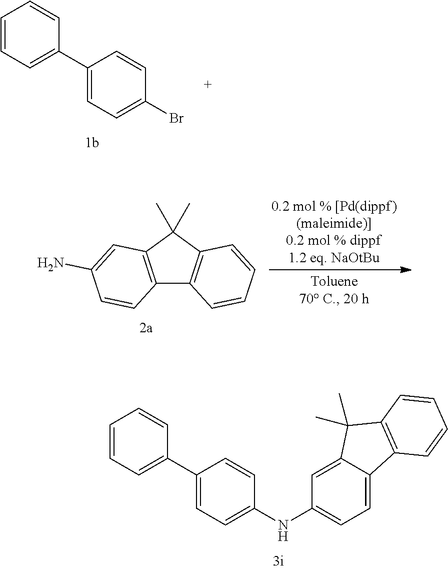 [SMILES: Brc1ccc(-c2ccccc2)cc1 ; CC1(C)c2ccccc2-c2ccc(N)cc21 ; CC1(C)c2ccccc2-c2ccc(Nc3ccc(-c4ccccc4)cc3)cc21]

According to the general protocol, compound 31 was prepared starting from 1b (259 mg, 1.00 mmol) and 2a (209 mg, 1.00 mmol) and was isolated by column chromatography (Al₂O₃, diethyl ether:hexane=1:2) in 89% yield (323 mg, 0.89 mmol). 1H NMR (400 MHz, chloroform-d): δ=7.72-7.63 (m, 4H), 7.59 (m, 2H), 7.51-7.44 (m, 3H), 7.37 (m, 2H), 7.32 (t, J=7.0 Hz, 1H), 7.25 (br. s, 1H), 7.22 (d, J=8.3 Hz, 2H), 7.13 (br. d, J=7.5 Hz, 1H), 5.92 (br. s, 1H), 1.54 ppm (s, 6H). ¹³C NMR (101 MHz, Chloroform-d): δ=155.3, 153.1, 142.7, 142.1, 140.8, 139.2, 133.5, 132.8, 128.7 (2C), 128.0 (2C), 126.9, 126.6, 126.5 (2C), 126.1, 122.4, 120.8, 119.1, 117.6 (2C), 117.2, 112.6, 46.8, 27.2 ppm (2C). CHN: Calculated for C₂₇H₂₃N: C, 89.71; H, 6.41; N, 3.87. found: C, 89.69; H, 6.27; N, 3.84.

### Example 4h

### Synthesis of Compound 3j

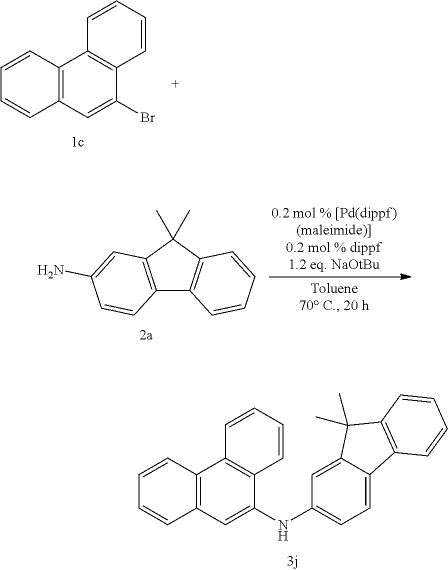 [SMILES: Brc1cc2ccccc2c2ccccc12 ; CC1(C)c2ccccc2-c2ccc(N)cc21 ; CC1(C)c2ccccc2-c2ccc(Nc3cc4ccccc4c4ccccc34)cc21]

According to the general protocol, compound 3j was prepared starting from 1c (268 mg, 1.00 mmol) and 2a (209 mg, 1.00 mmol) and was isolated by column chromatography (Al₂O₃, diethyl ether:hexane=1:2) in 87% yield (335 mg, 0.87 mmol). ¹H NMR (400 MHz, DMSO-d₆): δ=8.86 (m, 1H), 8.72 (m, 1H), 8.42 (s, 1H), 8.37 (dd, J=8.2 Hz, 1.1 Hz, 1H), 7.77-7.65 (m, 5H), 7.63 (s, 1H), 7.51 (m, 2H), 7.45 (d, J=7.5 Hz, 1H), 7.32 (d, J=2.0 Hz, 1H), 7.27 (dt, J=7.5 Hz, 1.1 Hz, 1H), 7.20 (dt, J=7.3 Hz, 1.0 Hz, 1H), 7.13 (dd, J=8.3 Hz, 2.0 Hz, 1H), 1.40 ppm (s, 6H), ¹³C NMR (101 MHz, DMSO-d₆): δ=154.7, 152.7, 144.6, 139.0, 137.8, 132.5, 131.0, 130.8, 127.5, 127.1, 127.1, 127.0, 126.9, 126.6, 126.4, 125.8, 124.5, 123.3, 123.3, 122.6, 122.5, 120.9, 118.9, 116.8, 112.4, 111.7, 46.3, 27.1 ppm (2C). CHN: Calculated for C₂₉H₂N: C, 90.35; H, 6.01; N, 3.63. found: C, 89.98; H, 6.31; N, 3.49.

### Example 41

### Synthesis of Compound 3k

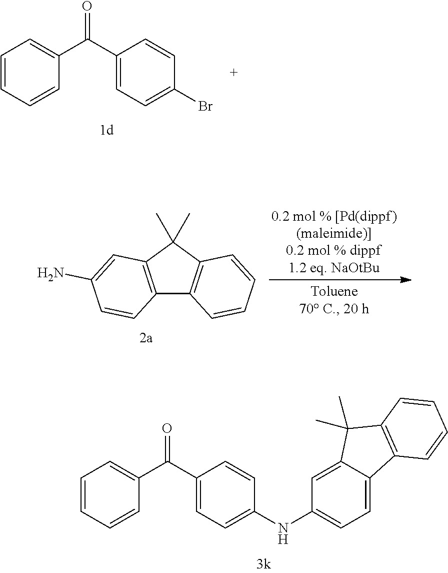 [SMILES: O=C(c1ccccc1)c1ccc(Br)cc1 ; CC1(C)c2ccccc2-c2ccc(N)cc21 ; CC1(C)c2ccccc2-c2ccc(Nc3ccc(C(=O)c4ccccc4)cc3)cc21]

According to the general protocol, compound 3k was prepared starting from 1d (269 mg, 1.00 mmol) and 2a (209 mg, 1.00 mmol) and was isolated by column chromatography (Al₂O₃, diethyl ether:hexane=2:1) in 76% yield (296 mg, 0.76 mmol). ¹H NMR (400 MHz, DMSO-d₆): δ=9.08 (s, 1H), 7.80-7.45 (m, 10H), 7.41-7.35 (m, 1H), 7.33-7.13 (m, 5H), 1.43 ppm (s, 6H), ¹³C NMR (101 MHz, DMSO-d₆): δ=193.6, 154.8, 152.9, 148.7, 140.6, 138.5 (2C), 132.9, 132.4 (2C), 131.5, 129.0 (2C), 128.3 (2C), 127.0, 126.7, 126.4, 122.6, 120.9, 119.3, 118.6, 114.1, 113.9 (2C), 46.4, 26.9 ppm (2C). CHN: Calculated for C₂₈H₂₃NO: C, 86.34; H, 5.95; N, 3.60. found: C, 86.05; H, 6.10; N, 3.52.

1.-16. (canceled)

17. A process for selective arylation of a primary aromatic amine of the formula A-NH₂ with an aromatic compound of the formula X—B to give a secondary aromatic amine A-NH—B, the radicals A and B independently of one another being identical or different, substituted or unsubstituted aromatic radicals and the radical X being a halogen or a trifluoromethylsulphonic acid radical, the aromatic carbon atoms in the secondary aromatic amine being bonded directly to the nitrogen atom, and at least one of the radicals, A or B, comprising a biphenyl unit, and the reaction being carried out in the presence of a base and a palladium complex, the palladium atom being complexed by at least one bis(dialkylphosphinoferrocene) ligand.

18. The process according to claim 17, wherein the alkyl substituents of the bis(dialkylphosphinoferrocene) ligands having two to five carbon atoms.

19. The process according to claim 17, wherein the alkyl substituents being selected from the group consisting of isopropyl, isobutyl, tert-butyl and combinations thereof.

20. The process according to claim 17, wherein both aromatic substituents, A and B, comprising a biphenyl unit, these units being identical to or different from one another.

21. The process according to claim 17, wherein the biphenyl unit being bonded directly to the secondary nitrogen atom of the amine.

22. The process according to claim 17, wherein the biphenyl unit being a bridged biphenyl unit of the formula 2 or 3 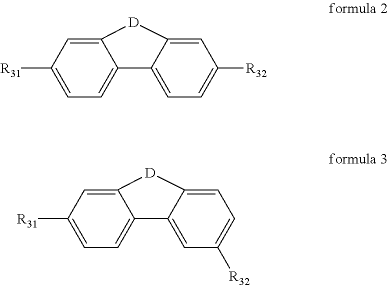
where D is oxygen, sulphur, nitrogen or carbon and is optionally substituted in the case of nitrogen singly or in the case of carbon doubly by methyl, ethyl, biphenyl, naphthyl or phenyl.

23. The process according to claim 17, wherein the biphenyl unit being 2-fluorene, 3-fluorene, 2-(9,9-diphenylfluorene), 2-(9,9-dimethylfluorene), 3-(9,9-diphenylfluorene), 3-(9,9-dimethylfluorene), 3-(4-phenyl)-9-phenyl-9H-carbazole, 3-(4-phenyl)-9-methyl-9H-carbazole, 3-(4-phenyl)-9-biphenyl-9H-carbazole, 2-(4-phenyl)-9-phenyl-9H-carbazole, 2-(4-phenyl)-9-methyl-9H-carbazole, 2-(4-phenyl)-9-biphenyl-9H-carbazole, 3-(4-biphenyl)-9-phenyl-9H-carbazole, 3-(4-biphenyl)-9-methyl-9H-carbazole, 3-(4-biphenyl)-9-biphenyl-9H-carbazole, 2-(4-biphenyl)-9-phenyl-9H-carbazole, 2-(4-biphenyl)-9-methyl-9H-carbazole, 2-(4-biphenyl)-9-biphenyl-9H-carbazole, 3-(9-phenyl-9H-carbazole), 3-(9-methyl-9H-carbazole), 3-(9-biphenyl-9H-carbazole), 2-(9-phenyl-9H-carbazole), 2-(9-methyl-9H-carbazole), 2-(9-biphenyl-9H-carbazole) or triphenylene.

24. The process according to claim 17, wherein the palladium complex being used in amounts of 0.01 mol % to 1.5 mol %, based on the total molar amount of both reactants, of the primary aromatic amine A-NH₂ and of the haloaromatic X—B.

25. The process according to claim 17, wherein the palladium complex being used as a solid, solution or powder mixture with a solid bis(dialkylphosphinoferrocene).

26. The process according to claim 17, wherein comprising the steps of:
providing the primary aromatic amine A-NH₂, the haloaromatic X—B, a suitable solvent and optionally a bis(dialkylphosphinoferrocene) in a reaction vessel;
adding a palladium complex in which the palladium atom is complexed by at least one bis(dialkylphosphinoferrocene) ligand, in the form of a solid or a solution;
heating the resulting reaction mixture in the reaction vessel;
isolating the reaction product, a secondary aromatic amine A-NH—B; and
optionally purifying the secondary aromatic amine A-NH—B.

27. The process according to claim 17, wherein the compounds 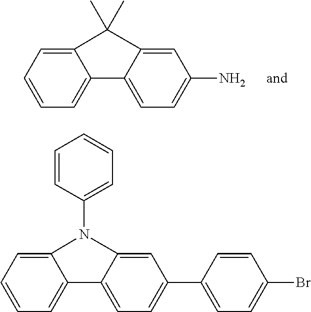
being present.

28. The process according to claim 17, wherein the palladium atom in the palladium complex being complexed additionally by 2,4,6,8-tetramethylcyclotetrasiloxane, bis(dibenzylideneacetone) or maleimide.

29. The process according to claim 17, wherein the palladium atom in the palladium complex being complexed by 1,1′-bis(diisopropylphosphino)ferrocene.

30. A chemical compound comprising 1,1′-bis(diisopropylphosphino)ferrocene-palladium(0)-maleimide [Pd(dippf)(maleimide)]; 1,1′-bis(diiso-propylphosphino)ferrocenepalladium(0)-1,3-divinyl-1,1,3,3-tetramethyldisiloxane [Pd(dippf)(VTS)]; 1,1′-bis(diisopropylphosphino)ferrocenepalladium(0)-bis(dibenzylideneacetone) [Pd(dippf)(dba)]; 1,1′-bis(diisopropylphosphino)ferrocene-palladium(0)-naphthoquinone [Pd(dippf)(naphthoquinone)]; 1,1′-bis(diisopropylphosphino)ferrocenepalladium(0)-maleic anhydride [Pd(dippf)(maleic anhydride)]; 1,1′-bis(diisopropylphosphino)ferrocenepalladium(0)-diethyl maleate [Pd(dippf)(diethyl maleate)]; or 1,1′-bis(diisopropylphosphino)ferrocenepalladium(0)-norbornene [Pd(dippf)(norbornene)].

31. A powder mixture comprising a bis(dialkylphosphinoferrocene) and a palladium complex in which the palladium atom is complexed by at least one bis(dialkylphosphinoferrocene) ligand.

32. A catalyst for Hartwig-Buchwald coupling which comprises the compound according to claim 30.

33. A catalyst for Hartwig-Buchwald coupling which comprises the powder mixture according to claim 31.
